(19) United States
(12) Patent Application Publication
(10) Pub. No.: US 2022/0148172 A1
(43) Pub. Date: May 12, 2022

(54) SYSTEM AND METHOD FOR DETECTING MEDICAL CONDITIONS

(71) Applicant: Henry Ford Health System, Detroit, MI (US)

(72) Inventors: Chad Louis Klochko, Royal Oak, MI (US); Steven Bishoy-Hanna Soliman, Ann Arbor, MI (US)

(21) Appl. No.: 17/519,353

(22) Filed: Nov. 4, 2021

Related U.S. Application Data
(60) Provisional application No. 63/111,858, filed on Nov. 10, 2020.

(51) Int. Cl. G06T 7/00; G16H 30/40; G16H 50/30; G06K 9/32; G06F 3/14

(52) U.S. Cl. CPC G06T 7/0012; G16H 30/40; G16H 50/30; G06T 2207/30008; G06F 3/14; G06K 2209/055; G06K 9/3233

(57) ABSTRACT

A method of detecting the presence or absence of a particular medical condition for a patient. The method comprises acquiring at least one image of an area of interest of the patient's body and identifying a first region of interest within the at least one acquired image corresponding to a first anatomical structure of interest and a second region of interest within the at least one acquired image corresponding to a second anatomical structure of interest. The method further comprises evaluating the first and second regions of interest, detecting the presence or absence of the medical condition based on the evaluation of the first and second regions of interest, and generating an electrical signal indicative of the detected presence or absence of the medical condition.

### CROSS REFERENCE TO RELATED APPLICATIONS

This application claims the benefit of U.S. Provisional Application No. 63/111,858 filed Nov. 10, 2020, the entire contents of which are hereby incorporated by reference.

### TECHNICAL FIELD

This disclosure relates generally to the detection of medical conditions in patients, and more particularly, to a system and method that uses one or more images of an area of interest of a patient's body to detect, predict, or otherwise determine the presence or absence of a particular medical condition for a patient (i.e., whether or not the patient has the medical condition).

### BACKGROUND

For many medical conditions, conventional ways of detecting the presence or absence of a medical condition for a patient (i.e., whether or not a patient has a medical condition) involve testing the patient's blood. Such medical conditions include diabetes and prediabetes. According to recent information provided by the United States Centers for Disease Control and Prevention, more than 34 million Americans have diabetes, with 30-32 million of those having type 2 diabetes. Of the 34 million people having diabetes, 26.9 million are diagnosed diabetic and 7.3 million are undiagnosed diabetic. Further, 88 million Americans aged 18 years or older are prediabetic. Diabetes may have any number of deleterious effects on a diabetic person's body. These include an increase in the risk of stroke and heart disease, kidney damage, and nerve damage, to cite only a few examples. And diabetes is a constant pandemic with a reported total cost in 2017 of over $327 billion dollars in the United States alone.

Testing for and/or detecting or determining whether or not a person has diabetes or is prediabetic are limited to testing the person's blood, and their blood sugar levels, in particular. Such tests include, for example, an HbA1c test that measures a person's average blood sugar over a number of months, a fasting plasma glucose (FPG) test that measures fasting blood sugar levels, and an oral glucose tolerance (OGTT) test that measures a person's blood sugar levels before and after the consumption of a glucose solution.

While blood tests such as those identified above have proven adequate for detecting whether a person is diabetic or prediabetic, they are not without their drawbacks. For example, recent studies have found that commonly used blood tests can misdiagnose patients and thus may not be an ideal screening method for diabetes. Additionally, a blood test can be an involved process that may include obtaining a sample of blood, sending the sample to a laboratory, analyzing or testing the sample, and then communicating the test results to one or more parties. As such, blood tests take time and are resource intensive. Further, in some geographic areas or regions, suitable tools for drawing blood and/or laboratories are simply not available or are not always easily accessible, and so performing a blood test may prove difficult. Finally, for some people, having blood drawn can be a traumatic experience that may lead to unneeded anxiety and/or uncomfortable side effects, for example, one or more of bruising, bleeding, lightheadedness, skin irritation, and soreness.

Accordingly, there is a need for a system and method for detecting medical conditions that minimizes and/or eliminates one or more of the above-identified deficiencies in conventional detection methodologies/techniques.

### SUMMARY

In at least some implementations, a method of detecting the presence or absence of a particular medical condition for a patient comprises acquiring at least one image of an area of interest of the patient's body and identifying a first region of interest within the at least one acquired image corresponding to a first anatomical structure of interest and a second region of interest within the at least one acquired image corresponding to a second anatomical structure of interest. The method further comprises evaluating the first and second regions of interest, detecting the presence or absence of the medical condition based on the evaluation of the first and second regions of interest, and generating an electrical signal indicative of the detected presence or absence of the medical condition.

In at least some implementations, a system for detecting the presence or absence of a particular medical condition for a patient comprises one or more electronic processors and one or more electronic memories wherein each of the one or more electronic memories is electrically connected to at least one of the one or more electronic processors and has instructions stored therein. The one or more electronic processors are configured to access the one or more electronic memories and to execute the instructions stored therein such that the one or more electronic processors are configured to: acquire at least one image of an area of interest of the patient's body; identify a first region of interest within the at least one acquired image corresponding to a first anatomical structure of interest and a second region of interest within the at least one acquired image corresponding to a second anatomical structure of interest; evaluate the first and second regions of interest; detect the presence or absence of the medical condition based on the evaluation of the first and second regions of interest; and generate an electrical signal indicative of the detected presence or absence of the medical condition.

In at least some implementations, a non-transitory, computer-readable storage medium storing program instructions thereon that, when executed on one or more electronic processors, causes the one or more electronic processors to carry out the method of: acquiring at least one image of an area of interest of the patient's body; identifying a first region of interest within the at least one acquired image corresponding to a first anatomical structure of interest and a second region of interest within the at least one acquired image corresponding to a second anatomical structure of interest; evaluating the first and second regions of interest; detecting the presence or absence of the medical condition based on the evaluation of the first and second regions of interest; and generating an electrical signal indicative of the detected presence or absence of the medical condition.

Further aspects or areas of applicability of the present disclosure will become apparent from the detailed description, claims and drawings provided hereinafter. It should be understood that the summary and detailed description, including the disclosed embodiments and drawings, are merely illustrative in nature intended for purposes of illustration only and are not intended to limit the scope of the invention, its application, or use. Thus, variations that do not depart from the gist of the disclosure are intended to be within the scope of the invention.

### BRIEF DESCRIPTION OF DRAWINGS

One or more aspects of the disclosure will hereinafter be described in conjunction with the appended drawings, wherein like designations denote like elements, and wherein:

FIG. 1 is a schematic and block diagram of an illustrative embodiment of a system for detecting the presence or absence of a medical condition for a patient;

FIG. 2 is a front view of an illustrative embodiment of an ultrasound system of which the system illustrated in FIG. 1 may be a part or with which the system illustrated in FIG. 1 may be used;

FIG. 3 a flowchart of an illustrative embodiment of a method that may be used to detect the presence or absence of a medical condition for a patient; and

FIG. 4 is an ultrasound image of a portion of a patient's body that may be used, for example, in the performance of one or more steps of the method illustrated in FIG. 3.

### DETAILED DESCRIPTION

The systems and methods described herein are configured to predict, detect, or otherwise determine the presence or absence of a medical condition (or that a medical condition is likely present or absent) for a patient using one or more images of an area of interest of the patient's body. In an embodiment, at least one image of the area of interest of the patient's body is acquired and then first and second regions of interest corresponding to respective anatomical structures of interest are identified in each of the acquired image(s). The identified regions are then evaluated and, based on that evaluation, the presence or absence of a medical condition the system and method are intended to detect can be detected.

In at least some embodiments, the systems and methods described herein are directed to artificial intelligence-driven detection of medical conditions using one or more machine learning models at one or more steps of the detection process. Accordingly, in at least some embodiments, the systems and methods described herein may employ artificial intelligence and machine learning techniques to detect the presence or absence of a particular medical condition.

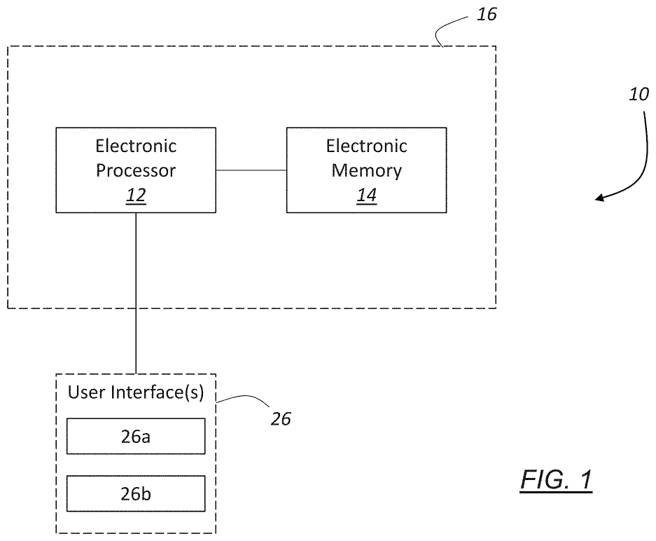

Referring now to the drawings, FIG. 1 shows an illustrative embodiment of a system 10 for predicting, detecting, or otherwise determining the presence or absence of a particular medical condition for a patient (i.e., detecting whether or not the patient has the particular medical condition). In an embodiment, the system includes, at least in part, one or more electronic processors 12 configured to execute instructions that are stored on or in one or more electronic memories 14 accessible by the one or more electronic processors 12.

Each of the one or more electronic memories 14 includes instructions, for example, computer or electronic instructions, that, when executed by the one or more processors 12, causes the one or more processors 12 to carry out one or more operations, such as, for example, one or more of the operations that are part of the method(s) described herein below. Each of the one or more processors 12, which may include one or more electrical inputs and one or more electrical outputs, may be any of a variety of devices capable of processing electronic instructions, including, for example and without limitation, microprocessors, microcontrollers, host processors, and application specific integrated circuits (ASICs). Each of the one or more processors 12 may be a dedicated processor used only for or by the system 10, or may be shared with other systems, and each of the one or more processors 12 may carry out various functionality in addition to that specifically described herein. Each of the one or more processors 12 may execute various types of digitally or electronically-stored instructions, such as, for example and without limitation, software, firmware, scripts, etc.

Each of the one or more electronic memories 14 may be any of a variety of electronic memory devices that can store a variety of data and information. This includes, for example, software, firmware, programs, algorithms, trained machine learning models, thresholds, scripts, and other electronic instructions and information that, for example, are required to perform or cause to be performed one or more of the operations or functions described elsewhere herein. Each of the one or more electronic memories 14 may comprise, for example, a powered temporary memory or any suitable non-transitory, computer-readable storage medium. Examples include, but are not limited to: different types of random-access memory (RAM), including various types of dynamic RAM (DRAM) and stage RAM (SRAM); read-only memory (ROM), solid-state drives (SSDs) (including other solid-state storage such as solid state hybrid drives (SSHDs)); hard disk drives (HDDs); magnetic or optical disc drives; or other components suitable for storing computer instructions used to carry out some or all of the various operations and/or functionality described herein.

In at least some embodiments, the aforementioned instructions may be provided as a computer program product, or software, that may include a non-transitory, computer-readable storage medium. This storage medium may have instructions stored thereon, which may be used to program a computer system (or other suitable electronic devices, for example, an electronic processor) to implement some or all of the functionality described herein, including one or more steps of the one or more methods described below. A computer-readable storage medium may include any mechanism for storing information in a form (e.g., software, processing application, etc.) readable by a machine (e.g., a computer or processing unit). The computer-readable storage medium may include, but is not limited to: magnetic storage medium (e.g., floppy diskette); optical storage medium (e.g., CD-ROM); magneto optical storage medium; read only memory (ROM); random access memory (RAM); erasable programmable memory (e.g., EPROM and EEPROM); flash memory; or electrical or other types of medium suitable for storing program instructions. In addition, program instructions may be communicated using optical, acoustical, or other form of propagated signal (e.g., carrier waves, infrared signals, digital signals, or other types of signals or mediums).

In any event, in an embodiment, the system 10 may include a single processor 12 and single memory 14 that is accessible by the processor 12. In such an embodiment, the functionality described herein that is attributable to an electronic processor may be performed or carried out by the processor 12, and the memory 14 may store the instructions required to carry out or perform such functionality. In other embodiments, however, the system 10 may include two or more electronic processors 12 and/or two or more memories 14, wherein each of the electronic memories 14 is accessible by one or more of the electronic processors 12. In an embodiment wherein the system 10 includes two or more electronic processors 12, the functionality described herein that is attributable to an electronic processor may be divided among the two or more processors 12 such that the functionality is performed or carried out by a collection of processors as opposed to a single processor. And in an embodiment wherein the system 10 includes two or more electronic memories 14, instructions needed to carry out or perform at least some of the functionality described herein may be divided among the two or more memories 14 such that different memories may be accessed by one or more processors to carry out or perform that functionality. Accordingly, it will be appreciated that the present disclosure is not intended to be limited to the system 10 including any particular number of electronic processors 12 and/or electronic memories 14. For purposes of illustration and clarity, however, the description below will be with respect to a non-limiting embodiment wherein the system 10 includes a single electronic processor 12 and a single electronic memory 14.

In an embodiment, the processor 12 and the memory 14 may comprise components of the system 10 that are separate and distinct from other components of the system 10, for example, the components described below. In other embodiments, however, the processor 12 and/or memory 14 may be part of or incorporated into another component of the system 10. For example, as shown in FIG. 1, the system 10 may include an electronic controller 16 that may include I/O devices and other known components. In such an embodiment, the processor 12 and/or memory 14 may be incorporated into the controller 16 and comprise constituent components thereof.

In addition to the components described above, the system 10 may further include one or more additional components or features, such as, for example and without limitation, a housing or enclosure within which the electronic processor 12 and/or the electronic memory 14 are disposed. Additional components the system 10 may include depends, at least in part, on the particular implementation of the system 10.

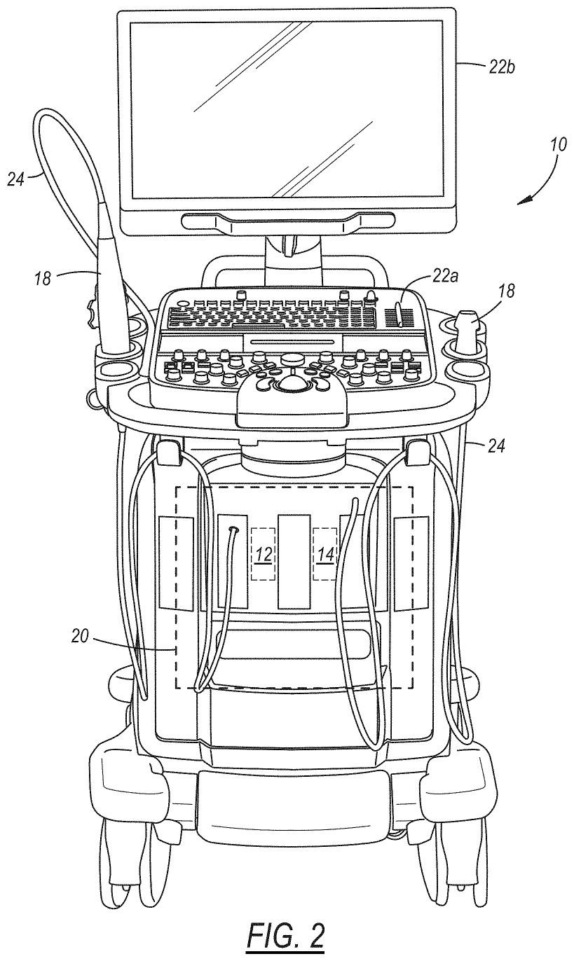

For example, in an embodiment, the system 10 comprises an ultrasound system such as, for example, that illustrated in FIG. 2. Such systems generally include, among potentially other components, one or more transducer probes 18, a central processing unit (CPU) 20, and one or more user interfaces 22. As is well known in the art, the transducer probe 18 is configured to emit and receive sound waves. The CPU 20, which is electrically connected to the transducer probe 18 by one or more cables 24, is the nerve center of the system and includes an electronic processor (e.g., electronic processor 12), and an electronic memory (e.g., electronic memory 14), and other circuitry and components needed for carrying out the operation and functionality of the system. For example, the CPU 20 may be configured to control the provision of electrical current to the transducer probe 18 to emit sound waves, and to receive electrical pulses generated in response to soundwaves or echoes received by the transducer probe 18. The CPU 20 may also be configured to process data and generate images that are displayed on one of the user interfaces 22.

As briefly mentioned above, the system 10 may also include one or more user interfaces 22. In an embodiment, each of the user interfaces 22 is electrically connected to the CPU 20 and is configured to permit one-way or two-way communication between the system 10 and a user (e.g., a medical professional). The user interfaces 22 may include any number of devices suitable to display or provide information to a user and/or to receive information from a user. For example, the one or more user interfaces 22 may comprise one or more of: a liquid crystal display (LCD); a touch screen LCD; a cathode ray tube (CRT); a plasma display; a keypad; a computer mouse or roller ball; one or more switches, buttons, or knobs; one or more indicator lights (e.g., light emitting diodes (LEDs)); a speaker; a microphone; a graphical user interface (GUI); a text-based interface; or any other display or monitor device. Among other things, the user interface(s) 22 may allow the user to exert a measure of control over the system 10. For example, in the embodiment illustrated in FIG. 2, one or more user interfaces 22 (user interface 22a) may allow the user to control parameters or characteristics of the ultrasound pulses generated by the system 10, for example, the frequency and duration of the pulses. The user interfaces 22 may also provide a way for a user to receive information or indications from the system 10 relating to, for example, the operation or functionality of the system 10. For example, one or more user interfaces 22 (user interface 22b in FIG. 2) may display images and/or other data generated by the system 10.

In any event, in an embodiment wherein the system 10 comprises an ultrasound system, the CPU 20 of the ultrasound system may comprise the electronic processor 12 and the electronic memory 14. Alternatively, one or both of the electronic processor 12 and the electronic memory 14 may be separate and distinct from the CPU 20, but may be electrically connected thereto.

While a particular ultrasound system or type of system is shown in FIG. 2 and described above, it will be appreciated that the system 10 may include other types of ultrasound systems known in the art. For example, the system 10 may include a handheld system that comprises a transducer probe configured to be electrically connected to a suitable handheld, portable, and/or mobile device, for example, smart phone, tablet, computer, PDA, etc. In such an embodiment, the handheld device may have a computer application or other software stored in or on an electronic memory thereof to allow the handheld device to serve as the CPU and/or user interface of the system.

While in the embodiment described above the system 10 comprises an ultrasound system, in other embodiments the system 10 may be separate and distinct system that is configured for use with an ultrasound system. More particularly, the system 10, and the electronic processor 12 thereof in particular, may be configured to be electrically connected to a component of an ultrasound system, for example, the CPU thereof, to obtain therefrom information needed for the electronic processor 12 to carry out or perform some or all of the operations or functionality described below (e.g., information in the nature of ultrasound images or ultrasound cine loops generated by the ultrasound system). In such an embodiment, the electronic processor 12 may also be configured to be electrically coupled or connected to one or more of the user interfaces of the ultrasound system to allow the user to receive information or indications from the ultrasound system relating to the operation or functionality of the system 10 and/or analyses performed by the system 10, for example, an indication as to whether the system 10 has detected the presence or absence of a particular medical condition for a patient, and thus, whether or not a patient has the particular medical condition. In such an embodiment, the system 10 may be configured to be electrically connected to the ultrasound system via one or more cables or electrical conductors, and/or may be wirelessly connected via a wireless electronic data connection, such as, for example and without limitation, via Bluetooth™ and/or Wi-Fi™.

In an implementation or embodiment of the system 10 wherein the system 10 does not comprise an ultrasound system, the system 10 may also include any number of additional components. For example, and as illustrated in FIG. 1, the system 10 may include one or more user interfaces 26 that are electrically connected to the electronic processor 12 of the system 10. As with the user interfaces 22 of the ultrasound system described above, each of the user interfaces 26 is configured to permit one-way or two-way communication between the system 10 and a user (e.g., a medical professional), and may include any number of devices suitable to display or provide information to a user and/or to receive information from a user. The one or more user interfaces 26 may include, for example, one or more of: a liquid crystal display (LCD); a touch screen LCD; a cathode ray tube (CRT); a plasma display; a keypad; a computer mouse or roller ball; one or more switches, buttons, or knobs; one or more indicator lights (e.g., light emitting diodes (LEDs)); a speaker; a microphone; a graphical user interface (GUI); a text-based interface; or any other display or monitor device. Among other things, the one or more of the user interface(s) 26 may allow the user to exert a measure of control over the system 10. For example, in the embodiment illustrated in FIG. 1, one or more user interfaces 26 (user interface 26a) may allow the user to control one or more operating parameters of the system 10. The same or other of the one or more user interfaces 26 may also allow for the user to receive information or indications from the system 10 relating to, for example, the operation or functionality of the system 10. For example, one or more user interfaces 26 (user interface 26b in FIG. 1) may display indications relating to an analysis performed by the system 10, including an indication as to whether or not the system 10 has detected that a patient has a particular medical condition.

While various implementations or embodiments of the system 10 have been described above, the present disclosure is not intended to be limited to any particular implementation. Rather, it will be appreciated that the system 10 may be implemented in any number of suitable ways that vary in one way or another from those described above, including comprising additional, alternative, or fewer components than the described implementations. Accordingly, the present disclosure is not intended to be limited to any particular implementation(s) of the system 10.

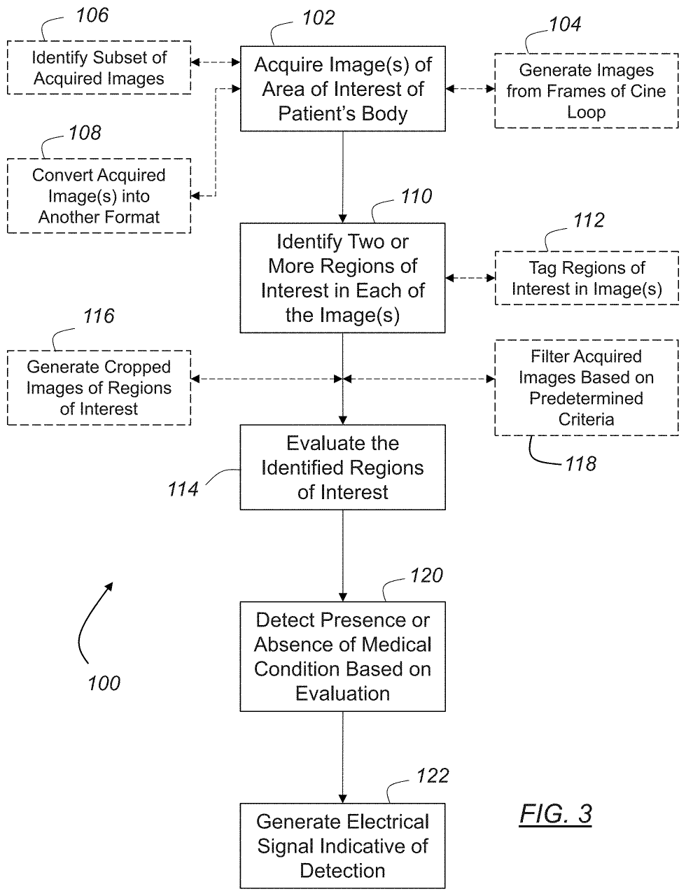

With reference to FIG. 3, there is shown a method 100 for detecting, predicting, or otherwise determining the presence or absence of a particular medical condition for a patient (i.e., detecting whether or not the patient has the medical condition). For purposes of illustration and clarity, method 100 will be described only in the context of the system 10 described above. It will be appreciated, however, that the application of the present methodology is not meant to be limited solely to such an implementation, but rather method 100 may find application with any number of implementations or embodiments of a system suitable for performing the methodology. It will be further appreciated that while the steps of method 100 will be described as being performed or carried out by one or more particular components of the system 10 (e.g., the electronic processor 12 and/or electronic memory 14), in other embodiments, some or all of the steps may be performed by suitable components of the system 10 other than that or those described. Accordingly, it will be appreciated that the present disclosure is not intended to be limited to an embodiment wherein particular components are configured to perform any particular steps. Moreover, it will be appreciated that unless otherwise noted, the performance of method 100 is not meant to be limited to any one particular order or sequence of steps; rather the steps may be performed in any suitable and appropriate order or sequence and/or at the same time.

It is contemplated that the method 100 may be used for detecting, predicting, or otherwise determining whether or not a patient has one or more of a variety of medical conditions. These medical conditions may include, for example and without limitation, one or more of: diabetes (e.g., type 2 diabetes); prediabetes; muscle atrophy/fatty infiltration (e.g., atrophy/fatty infiltration of rotator cuff muscles); and steatosis of the liver, to cite just a few examples. Although the method 100 may be applicable to detecting the presence or absence of a number of different medical conditions, for purposes of illustration and clarity, the description below will primarily be with respect to the use of the method 100 for detecting the presence of diabetes, that is, detecting whether or not a patient has diabetes. However, it should be understood that the various teachings described herein could be applied for detecting the presence of any number of other medical conditions, and as such, the present disclosure is not intended to limited to the use of the method 100 for detecting any particular medical condition(s).

In any event, in an embodiment, the method 100 includes a first step 102 of acquiring at least one image of an area of interest of a patient's body. In an embodiment, an electronic processor, for example, the electronic processor 12 of the system 10, is configured to acquire the image(s), and the image(s) may be acquired in a number of ways. For example, in one embodiment, one or more images generated by an imaging system used to perform or conduct a study of the patient's body and stored in an electronic memory may be obtained. The electronic memory may comprise, for example, the electronic memory 14 of the system 10 or another memory separate and distinct from the system 10, for example, an electronic memory of the imaging system that generated the image(s). In such an embodiment, the electronic processor would access the appropriate electronic memory and obtain the image(s). In another embodiment, rather than obtaining the image(s) from an electronic memory, images generated during a study may be provided directly to the electronic processor performing step 102 by an imaging system, or may be generated by the electronic processor itself, and as such, the electronic processor may acquire the image(s) from the component that generates the image(s) or may generate the images itself.

In an embodiment, the image(s) acquired in step 102 comprise ultrasound images generated by an ultrasound system during an ultrasound study of the area of interest of the patient's body. In such an embodiment, the images may be two-dimensional grayscale images. Additionally, the images may be still images and/or may be images generated from individual frames of a cine loop generated during the study. In the latter instance, step 102 may further include a substep 104 of generating or creating one or more images from the cine loop by, for example, converting some or all of the frames of the cine loop into an individual image using any suitable known image processing technique(s). While in an embodiment ultrasound images are acquired in step 102, in other embodiments images other than ultrasound images may be acquired in step 102. For example, it is contemplated that images from other imaging modalities, for example, computed tomography (CT), magnetic resonance imaging (MRI), and/or other suitable modalities may be additionally or alternatively acquired in step 102.

The number of images acquired in step 102 is dependent, at least in part, on the particular implementation or embodiment of the method 100. For example, in some implementations, a single image may be acquired in step 102 and used in subsequent steps of the method 100. In other implementations or embodiments, however, a plurality of images may be acquired in step 102. For example, all or a subset of the images from a previously conducted study may be acquired in step 102. In an embodiment wherein less than all of the images from a study are acquired, step 102 may include a substep 106 comprising identifying a subset of images. Alternatively, rather than the subset of images being identified in step 102, in other embodiments, method 100 may include a step performed prior to step 102 that comprises identifying a subset of images that are subsequently acquired in step 102.

In any event, if applicable, the subset of images may be identified using one or more desired filtering criteria. For example, in an embodiment, all of the still images and only a certain percentage of images generated from frames of a cine loop of a study (e.g., the images corresponding to the middle X % of the frames of the cine loop) may be stored in an electronic memory, and the rest of the images may be discarded or at least segregated from those images. Alternatively, the subset of images may be identified manually or using other desired filtering criteria (e.g., only still images from certain points in time of the study, images corresponding to every Y number of frames in the cine loop, etc.) Regardless of how the images are selected or identified for the subset of images, the images of the subset of images are saved in an electronic memory and may be acquired or obtained therefrom.

Accordingly, it will be appreciated in view of the above that any number of images may be acquired in step 102, and as such, the present disclosure is not intended to be limited to the acquisition of any particular number(s) of images.

In some embodiments, images in a native format may be acquired in step 102 and used in one or more subsequent steps of the method 100. For example, in some embodiments, images in the Digital Images and Communications in Medicine (DICOM) format may be acquired in step 102. In other embodiments, however, the acquired images must first be converted from a native format (e.g., DICOM) to a single image format, for example and without limitation, a portable network graphics (png or .png) format, a joint photographic experts group (jpeg or .jpeg or jpg) format, or another suitable format. In such an embodiment, step 102 may include a substep 108 in which images are converted to a suitable format. The images requiring conversion may be converted using known image processing techniques that may employ a combination of an image processing script (e.g., a python script) suitable for analyzing pixel/image data, and an appropriate library, for example, the pydicom library. Alternatively, rather than images being converted in substep of step 102, in other embodiments, method 100 may include a step performed prior to step 102 that comprises converting the images that are subsequently acquired in step 102.

As briefly described above, the image or images acquired in step 102 are of an area of interest of the patient's body. Which area of the patient's body is the area of interest depends on the medical condition(s) the method is intended to detect. For example, where the medical condition comprises diabetes or prediabetes, the area of interest may comprise an area of the patient's body that includes one of the patient's shoulders including the deltoid muscle. Where the medical condition is atrophy/fatty infiltration of a rotator cuff, the area of interest comprises an area of the patient's body that includes one of the patient's rotator cuff muscles (e.g., the supraspinatus muscle) and, in some embodiments, the scapular cortex. And wherein the medical condition comprises steatosis of the liver or other types of liver damage, the area of interest comprises an area of the patient's body that includes the patient's liver. Accordingly, it will be appreciated that the present disclosure is not intended to be limited to the area of interest of the patient's body being any particular area(s).

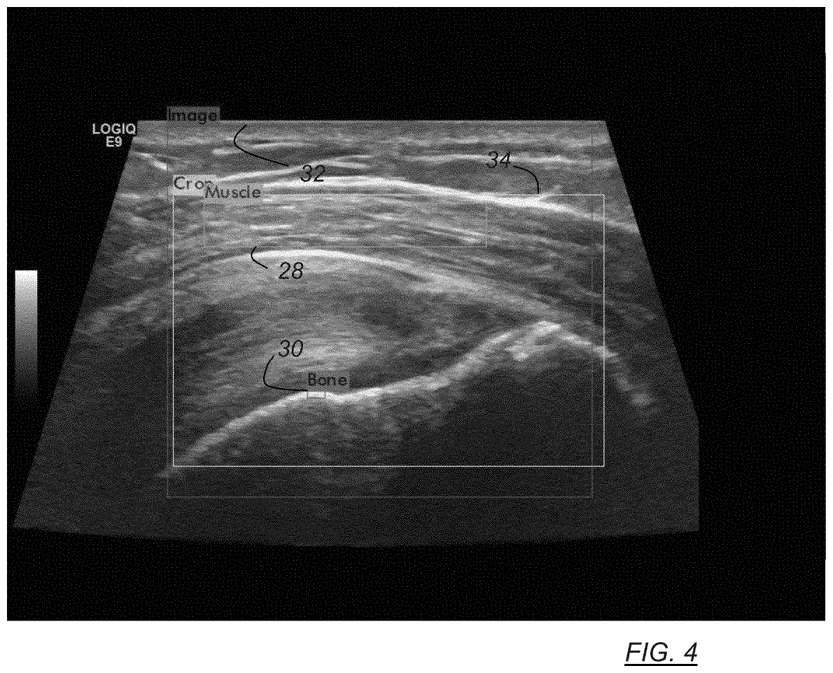

Once the image or images are acquired in step 102, the method moves to a step 110 of identifying two or more regions of interest in the image or each of the images. With reference to FIG. 4, in an illustrative embodiment, step 110 comprises identifying a first region of interest 28 corresponding to a first anatomical structure of interest and a second region of interest 30 corresponding to a second anatomical region of interest. In an embodiment, the first anatomical region of interest corresponding to the first region of interest 28 may comprise a patient's muscle, and the second anatomical structure corresponding to the second region of interest 30 may comprise a patient's bone/cortical bone. By way of example, in an instance wherein the medical condition the method 100 is intended to detect is diabetes or prediabetes, the first anatomical structure of interest may comprise a patient's deltoid muscle and so the first region of interest 28 corresponds to the patient's deltoid muscle, while the second anatomical structure of interest may comprise a patient's cortical bone and so the second region of interest corresponds to the patient's cortical bone. Similarly, in an instance wherein the medical condition the method 100 is intended to detect is atrophy/fatty infiltration of a rotator cuff muscle, the first anatomical structure of interest may comprise a patient's supraspinatus muscle and so the first region of interest 28 corresponds to the patient's supraspinatus muscle, while the second anatomical structure of interest may comprise a patient's scapular cortex and so the second region of interest corresponds to the patient's scapular cortex.

While in the embodiment described above only two regions of interest are identified in step 110, in other embodiments more than two regions of interest may be identified. For example, in some embodiments additional regions of interest corresponding to different anatomical structures of interest may be identified, for example, regions corresponding to tendons, organs, muscles, nerves, and bone other than those anatomical structures corresponding to the first and second identified regions of interest 28, 30. Additionally, or alternatively, in some embodiments, regions of interest corresponding to different portions of the same anatomical structure that correspond to the first and/or second identified regions of interest 28, 30 may be identified. For example, in an instance where the first region of interest 28 corresponds to a patient's muscle, step 110 may comprise identifying one or more additional regions of interest corresponding to different portions of the same muscle. Similarly, in an instance where the second region of interest 30 corresponds to a patient's bone, step 110 may comprise identifying one or more additional regions of interest corresponding to different portions of the same bone.

In still other embodiments, one or more regions of interest may additionally or alternatively be identified that do not directly correspond to any particular anatomical structures of the patient's body per se, but rather correspond to desired portions of the image itself. One example of such a desired portion of the image is that corresponding to largest area of the image that is in the diagnostic portion of the image (e.g., the largest area of the image that both contains anatomical structures but does not include any text). Accordingly, in some embodiments such as that illustrated in FIG. 4, step 110 comprises identifying a region of interest 32 corresponding to the largest area of the image that is in the diagnostic portion of the image to avoid, for example, any areas of artifact. Another example of a desired portion of the image is the smallest or tightest portion of the image that includes other identified regions of interest, for example, those corresponding to anatomical structures of interest (e.g., the first and second regions of interest 28, 30). Accordingly, in some embodiments, such as, for example, that illustrated in FIG. 4, step 110 comprises identifying a region of interest 34 corresponding to the smallest or tightest portion of the image that includes other identified regions of interest.

In view of the foregoing, it will be appreciated that any number of regions of interest may be identified in step 110, and thus, it will be further appreciated that the present disclosure is not intended to be limited to any particular region or number of regions.

The identification of regions of interest in step 110 may be carried out or performed in a number of ways. One way is by a user manipulating a user interface, for example, one of the user interfaces 26 of the system 10, to manually identify the regions interest. More particularly, a user may view the image on a display or monitor and then manipulate a user interface (e.g., keyboard, mouse, etc.) to place a box or other geometric shape around a desired portion of the image, thereby defining that region or portion of the image as a region of interest. Once the regions of interest are manually defined, an electronic processor, for example, the electronic processor 12 of the system 10, may be configured to determine or predict coordinates of each of the regions of interest within the image. Those coordinates may then be saved in a file corresponding to or associated with that particular image (e.g., a .txt file) that is stored in or on an electronic memory, for example, the memory 14 of the system 10. In an embodiment, the coordinates may comprise, for example, the x-center of the region of interest, the y-center of the region of interest, and the lengths of the x and y segments of the region of interest as respective percentages of the length and height relative to the overall image. Additional or alternative coordinates may include, for example and without limitation, the coordinates of a polygon or other shape outlining the region of interest, a pixel map that traces the exact border of the anatomy/region of interest, or other suitable coordinates.

Additionally, or alternatively, one or more, and in an embodiment, all, of the regions of interest may be identified by an electronic processor, for example, the electronic processor 12 of the system 10. For example, in some embodiments, step 110 may comprise applying a machine learning model or algorithm trained to perform image recognition to the or each of the acquired images to identify the regions of interest. More particularly, the trained machine learning model may be trained to recognize one or more desired portions of the image and/or features contained in the image to identify the desired regions of interest. In such an embodiment, once the machine learning model recognizes a portion or feature of the image it is trained to recognize, a bounding box corresponding to the region of interest may be calculated or defined, and coordinates of the region of interest within the image may be predicted or otherwise determined and saved in an electronic memory, for example, the memory 14 of the system 10.

By way of illustration, in an embodiment wherein the first and second regions of interest 28, 30 identified in step 110 correspond to first and second anatomical structures of interest, respectively, the trained machine learning model may be trained to recognize the first and second anatomical structures of interest in the image and to identify the region of interest within the image corresponding to those anatomical structures. Once the first and second anatomical structures of interest are recognized, bounding boxes containing portions of the image that include the first and second anatomical structures of interest may be calculated or defined to identify the regions of interest, and coordinates of the bounding boxes/regions of interest may be determined or predicted and saved in an electronic memory, for example, the memory 14 of the system 10. The same process may be followed for each anatomical structure of interest and/or each desired portion of an image that corresponds to a region of interest.

As will be appreciated by those of ordinary skill in the art, any number of trained machine learning models may be used to perform the functionality/operation described above. Suitable machine learning models or algorithms may include, but are certainly not limited to: deep learning models; trained neural networks (e.g., convolutional neural networks (CNN)); and object classification models/algorithms. For purposes of illustration only, one particular model or algorithm that may be used is the YOLOv5 model architecture; though other suitable models or algorithms may certainly be used instead (e.g., YOLOv4).

Regardless of the particular trained machine learning model or algorithm that is used to identify the regions of interest in step 110, the model may be trained using techniques well known in the art. While the particular way the model is trained may be model-dependent, in general terms, a set of images (i.e., training images) are fed to the model. These images may be tagged or otherwise marked to identify and show the anatomical structures of interest and/or desired portions of the image that is/are to be recognized. The model then learns the anatomical structures of interest and/or desired portions and works to recognize them using a second set of images (i.e., test images) that may or may not include one or more of the first set of training images. Based on the performance of the model with the test images, parameters of the model (e.g., biases, weights, etc.) may be adjusted or tuned to improve performance.

In any event, regardless of how the regions of interest are identified, after each region is identified, or after all of the regions of interest in a given image are identified, step 110 may comprise a sub step 112 of automatically tagging the image to indicate which regions of interest are contained in the image. For example, in an instance wherein a first region of interest corresponding to a first anatomical structure of interest is identified in step 110, the image may be tagged with a tag indicating that the image includes the first region of interest. Similarly, in an instance wherein a region of interest corresponding to a desired portion of the image that does not directly correspond to any particular anatomical structure of interest is identified in step 110, the image may be tagged with a tag indicating that the image includes that region of interest. The tags may take any suitable form. In an embodiment, each tag comprises a number that has been assigned to it and that represents a respective region of interest. For example, in an embodiment, a first tag comprises the number “0” and was previously assigned to and represents a region of interest that includes a first particular anatomical structure (e.g., a muscle), a second tag comprises the number “1” and was previously assigned to and represents a region of interest that includes a second particular anatomical structure (e.g., bone), etc. Regardless of the form the tags take and the particular tags the image is tagged with, the tags may be stored or saved in a file corresponding to or associated with that particular image (e.g., a .txt file) that is stored in or on an electronic memory, for example, the memory 14 of the system 12. In an embodiment, the tagging of the images may be done manually by the user. In other embodiments, however, the tagging may be done by an electronic processor, for example, the electronic processor 12 of the system 10.

It will be appreciated that in an embodiment wherein a single image is acquired in step 102, step 110 is performed for only that image. However, in an embodiment wherein multiple images are acquired in step 102, step 110 may be performed for more than one of the acquired images (e.g., all of the acquired images or at least a given subset thereof). In the latter instance, in at least some embodiments, step 110 is performed for each of the images before moving on to further steps of the method. In other embodiments, however, after step 110 is performed for one acquired image, one or more further steps of the method may be performed prior to step 110 being performed for another one of the acquired images.

In any event, once the desired regions of interest are identified in step 110, the method 100 moves on to a step 114 of evaluating the identified regions of interest. In an embodiment, step 114 comprises calculating a score of the image based on a given parameter of the identified regions of interest. In some embodiments, the score calculated in step 114 comprises a ratio of the given parameter of one or more regions of interest to the given parameter of one or more other regions of interest. For example, in an instance such as that described above wherein step 110 comprises identifying the first and second regions of interest 28, 30, step 114 comprises calculating a ratio of a given parameter of the first region of interest 28 to the given parameter of the second region of interest 30. In an embodiment, the ratio is calculated automatically by an electronic processor, for example, the electronic processor 12 of the system 10. Accordingly, the electronic processor may be configured to first determine the parameter(s) of interest of for each region of interest, and then calculate the ratio.

The particular parameter for which the score or ratio is calculated is dependent on the medical condition the method is intended to detect. For example, in an instance where the medical condition is diabetes or prediabetes, the parameter may be pixel intensity (e.g., grayscale pixel intensity in the instance where the images acquired in step 102 are ultrasound images). More specifically, in an embodiment wherein the images acquired in step 102 are ultrasound images, the score may be the ratio of the echogenicity or average grayscale pixel intensity of the first region of interest to the echogenicity or average pixel intensity of the second region of interest. So, in an embodiment wherein the first region of interest 28 corresponds to a muscle of the patient (e.g., deltoid muscle) and the second region of interest 30 corresponds to a bone of a patient, the score would be the ratio of the echogenicity or average pixel intensity of the first region of interest 28 corresponding to muscle to the echogenicity or average pixel intensity of the second region of interest 30 corresponding to bone.

In an embodiment wherein the parameter is the average pixel intensity and the score is the ratio of the average pixel intensity of a first region of interest to the average pixel intensity of a second region of interest, the average pixel intensity of a region of interest may be calculated by determining the intensity of each pixel in the region of interest, adding the pixel intensities of all of the pixels together, and then dividing the resulting total pixel intensity by the number of pixels in the region of interest.

While the description thus far has been with respect to determining a score comprising a ratio of a parameter of one region of interest to the parameter of one other region of interest, in some embodiments, more than two regions of interest may be used to determine the score/ratio. For example, in some embodiments, the average parameter of two or more regions of interest may first be determined, and the ratio may be the average parameter of those regions of interest to the parameter of a third region of interest (or the average parameter of the third region of interest and one or more other regions of interest of the same class). For purposes of illustration, in an instance wherein pixel intensity is the parameter of interest, the average pixel intensity of a first region of interest and the average pixel intensity of a second region of interest may be determined. The average pixel intensities of those regions of interest may then be used to determine an overall average pixel intensity of the two regions. A ratio may then be determined of the overall average pixel intensity of the first and second regions to the pixel intensity of a third region of interest. So, in an embodiment wherein the first region of interest corresponds to a muscle of the patient (e.g., deltoid muscle), the second region of interest corresponds to a cortical bone of the patient, and a third region of interest corresponds to a different portion of the bone of the patient, the overall average pixel intensity of the second and third regions of interest may first be determined, and then a ratio of the average pixel intensity of the second and third regions of interest to the average pixel intensity of the first region of interest may be calculated or determined.

In some embodiments, the method 100 may include one or more steps performed after the regions of interest are identified in step 110 and before the regions of interest are evaluated in step 114, and that may comprise optional steps. For example, in an embodiment, the method 100 may include a step 116 of processing the or each acquired image using, for example, one or more scripts (e.g., python script(s)) and the previously-predicted or determined coordinates of regions of interest to generate or create new or cropped images of and for at least certain of the identified regions of interest. By way of example, in an embodiment wherein first and second regions of interest identified in step 110 correspond to first and second anatomical structures of interest, respectively, step 116 may comprise locating the first region of interest using the previously-predicted or determined coordinates of the first region of interest, and then generating or creating a first new or cropped image corresponding to the first region of interest. Similarly, step 116 may further comprise locating the second region of interest using the previously-predicted or determined coordinates of the second region of interest, and then generating or creating a second new or cropped image corresponding to the second region of interest. In such an embodiment, the new or cropped images of the first and second regions may be evaluated in step 114, including, for example, using the cropped images to calculate the score for the original acquired image as described above. So, in an embodiment wherein first region of interest, and thus, a first cropped image corresponds to a muscle of the patient (e.g., deltoid muscle), the second region of interest, and thus, a second cropped image corresponds to a bone of a patient, and average pixel intensity is the parameter for which a score/ratio is calculated, the ratio would be the average pixel intensity of the first cropped image to the average pixel intensity of the second cropped image. In an embodiment wherein more than two regions of interest are used to determine the ratio, cropped images of each of those regions of interest may be generated or created and then used to determine the score or ratio among the two or more regions in the same or similar manner described above.

In an embodiment wherein the method includes step 116, the new or cropped images generated or created in substep 116 may be saved in an electronic memory with the corresponding original image. In other embodiments, however, the cropped images are not saved but rather are processed and used to calculate the score/ratio in step 114 without first saving the cropped images.

In an embodiment wherein multiple images are acquired in step 102, the evaluating step 114 may be performed for each of the acquired images such that a score is calculated for each acquired image. The scores of the images may then be combined using a statistical combination to determine an overall score for the collection of images. In an embodiment, the overall score comprises an average score for the collection of images that may be calculated from the individual scores of each of the acquired images. Accordingly, if there are N number of images, step 114 comprises calculating N scores and then calculating an overall average score for the collection of N images by adding all of the individual image scores together and dividing by the number of images for which scores were calculated (i.e., N). Similarly, in an instance wherein the images acquired in step 102 are taken along multiple imaging planes (e.g., one or more images along a long axis and one or more images along a short axis), scores may be calculated for each individual image and then an average score may be calculated for each imaging plane from those individual scores (e.g., an average score of the long axis and an average score for the short axis). The average scores for the different imaging planes may then be combined using a statistical combination (e.g., statistical mapping) to determine an overall score for the collection of acquired images.

In an embodiment wherein multiple images are acquired in step 102, and whether or not the method 100 includes the step 116 described above, the method 100 may include a step 118 of filtering the images acquired in step 102 using predetermined criteria to potentially restrict the number of images that are used in the evaluation step 114. In embodiment, the filtering in step is based on the identification of regions of interest in step 110, and, in particular, whether certain regions of interest or a certain number of regions of interest were identified.

For example, in an embodiment wherein the identifying step comprises identifying first and second regions of interest, the criteria used in step 118 may be that both the first and second regions of interest corresponding to first and second anatomical structures of interest were, in fact, identified. This may be determined by an electronic processor, for example, the electronic processor 12 of the system 10, checking the tags associated with the images in substep 112 of step 110 to determine if both the first and second regions of interest were, in fact, identified in the image (i.e., verifying that the image includes tags for both the first and second regions of interest). Similarly, in some embodiments, the criteria may be that the first and second regions of interest corresponding to one or more anatomical structures of interest, and one or more other regions of interest corresponding to desired portions of the image, were, in fact, identified. Again, this may be determined by an electronic processor, for example, the electronic processor 12 of the system 10, checking the tags associated with the images in substep 112 of step 110 to determine if all of the required regions of interest were, in fact, identified (i.e., verifying that the image includes tags for all of the required regions of interest). In any event, if it is determined that the required criteria is/are met, then the image may be evaluated in step 114. If, however, it is determined that the criteria is/are not met, the image may be discarded or at the very least not processed or evaluated in step 114.

Whether or not the method 100 includes one or both of steps 116 and 118 described above, following the evaluating step 114, the method 100 moves to a step 120 of detecting, predicting or otherwise determining the presence or absence of (i.e., whether or not patient has or likely has) the medical condition(s) the method is intended to detect. In an embodiment wherein a score is determined in step 114 for one or a collection of images acquired in step 102, step 120 may comprise detecting the presence of the medical condition (or lack thereof) using the score. This may comprise, for example, looking up the score in a pre-populated, empirically-derived look-up table or other data structure that correlates calculated scores with indications as to whether a medical condition is present or at least likely to be present. For example, assume that the score calculated in step 114 has a value of X. An electronic processor, for example, the electronic processor 12 of the system 10, may be configured to look up this value in a data structure stored in an electronic memory, for example, the memory 14 of the system 10, and determine whether that value corresponds to an indication of “condition detected or present” or “condition not detected or not present,” or some variant thereof.

In another embodiment, the score calculated in step 114 may be compared with one or more predetermined, empirically-derived threshold values stored in an electronic memory to detect the presence (or absence) of a medical condition. For example, assume again that the score calculated in step 114 is X. An electronic processor, for example, the electronic processor 12 of the system 10, may be configured to compare this value to a threshold value stored in an electronic memory, for example, the memory 14 of the system 10. Based on that comparison (i.e., whether the value is above (or, in an embodiment, equal to or above) or below (or, in an embodiment, equal to or below) the threshold), the electronic processor may detect the presence or absence of the medical condition.

In some embodiments, multiple thresholds may be stored in an electronic memory and one or more of those thresholds may be selected for use in step 120. For example, predetermined empirically-derived thresholds may be determined for different patient demographics or characteristics, such as, for example, body type, body mass index (BMI), weight, height, race or ethnicity, age, gender, etc., or a combination of two or more thereof (e.g., a threshold may be determined for an obese (body type) male (gender)). Based on the characteristics of the patient, one or more of these thresholds may be selected and used in step 120. In such an embodiment, the method may include a step of receiving or obtaining the relevant patient demographic information or characteristics that would be used to select the appropriate threshold (i.e., that or those characteristics upon which the thresholds are based), and then using that received or obtained information to select the appropriate threshold. This step may comprise part of a prior step of the method (e.g., step 102) or may be a separate step performed before or after one or more of the steps described above. In any event, the patient information may be received or obtained in a number of ways known in the art. One way is by receiving an input from a user interface device, for example, one or more of the user interfaces 26 of the system 10. That is, a user interface (e.g., a keyboard, a touch screen, a mouse, etc.) may be used to enter or select or input the relevant information and then that information may be received by, for example, an electronic processor electrically connected to the user interface, for example, the electronic processor 12 of the system 10. In other embodiments, the relevant information may be stored in or on an electronic memory of the system, for example, the electronic memory 14 of the system 10 (e.g., as part of or contained in a patient record stored in the memory). An electronic processor, for example, the electronic processor 12 may be configured to access that electronic memory and obtain or acquire the relevant information therefrom. Accordingly, it will be appreciated that the relevant patient information may be received or obtained in any number of ways, and as such, the present disclosure is not intended to be limited to any particular way(s) of doing so. Regardless of how the information is received or obtained, once it is received or obtained it may be used by an electronic processor to select the appropriate threshold to be used as described elsewhere herein.

Additionally, or alternatively, multiple thresholds may be stored in an electronic memory that correspond to different medical conditions. In such an embodiment, the score calculated in step 114 may be compared to each of the thresholds to determine or detect the presence or absence of the medical condition(s) corresponding to those thresholds.

For purposes of illustration only, in an instance wherein diabetes or prediabetes is the medical condition the method 100 is intended to detect, assume the following predetermined thresholds were empirically derived for different types of patients—0.35 for non-obese non-diabetic, 0.42 for obese non-diabetic, 0.48 for non-obese diabetic, and 0.54 for obese diabetic. Assume further that the score calculated in step 114 is 0.34, and that the patient is non-obese. When the score is compared to the predetermined thresholds, it can be determined or detected that diabetes is not present. It will be appreciated that while certain threshold values are provided above, they are provided for illustrative purposes only and that other suitable threshold values may certainly be used in addition to or instead of those identified above.

In addition to detecting the presence or absence of a medical condition, in at least some embodiments, and depending on the particular medical condition (e.g., atrophy/fatty infiltration of rotator cuff muscle), step 120 may also include assigning an indication or grade as to the severity of the condition (e.g., mild, moderate, severe). In at least some embodiments, the score calculated in step 114 may be used to assign such a grade. For example, one or more predetermined, empirically-derived thresholds or threshold ranges, each corresponding to a particular grade (e.g., mild, moderate, severe) may be stored in an electronic memory and may be used along with the calculated score to assign a grade to the medical condition. In such an embodiment, an electronic processor, for example, the electronic processor 12 of the system 10, may be configured to compare the score calculated in step 114 to one or more threshold values stored in an electronic memory, for example, the memory 14 of the system 10. Based on that comparison (i.e., whether the score is above (or, in an embodiment, equal to or above) or below (or, in an embodiment, equal to or below) the threshold(s)), the electronic processor may determine the severity of the condition and assign an appropriate grade accordingly. It will be appreciated that while one particular way of assigning a grade has been described, the present disclosure is not intended to be limited to any particular way(s), rather any suitable way be used.

In any event, following step 120, the method 100 may proceed to a step 122 of generating an electrical signal indicative or representative of the detection of the presence or absence of the medical condition in step 120. This step may be performed by the electronic processor that performed the detection step 120 or another processor. The signal generated in step 122 may be output to one or more components (e.g., a user interface 26 of the system 10) to provide an indication to the user as to whether or not the medical condition was detected. In an embodiment, this may comprise outputting the electrical signal to a user interface, such as, for example, a visual display or monitor to cause the user interface to display a visual indication as to whether or not the medical condition was detected in step 120. In other embodiments, the electrical signal may be output to a speaker or other user interface suitable to display or provide an indication as to whether the medical condition was detected in step 120.

While in the embodiment of the method 100 described above the evaluating step 114 and the detecting step 120 utilize a score to detect or determine the presence or absence of a medical condition, in other embodiments the evaluating step 114 and detecting step 120 may alternatively comprise using a trained machine learning model to evaluate the regions of interest identified in step 110 and to detect the presence or absence of the medical condition based thereon.

More specifically, in an embodiment, the evaluating step 114 and detecting step 120 may comprise applying a machine learning model or algorithm trained to perform image recognition to an image acquired in step 102, or one or more portions thereof, to predict, detect, or otherwise determine, based on the identified regions of interest, the presence or absence of the medical condition the method 100 is intended to detect. More particularly, the trained machine learning model may be trained to recognize the regions of interest identified in step 110 and the differences in one or more characteristics or parameters thereof to predict, detect, or otherwise determine the presence or absence of the medical condition. In some embodiments, the model may also assign to the determination, detection, or prediction made a confidence level of or in the prediction, detection, or determination or an indication as to the severity of the medical condition, in the event the medical condition is determined, detected, or predicted to be present.

For example, in an instance wherein the method 100 is intended to detect diabetes or prediabetes, and first and second regions of interest of an acquired image identified in step 110 correspond to first and second anatomical structures of interest, respectively, the trained machine learning model may be configured to detect the presence or absence of diabetes (i.e., whether or not the patient has diabetes) based on the relative difference in the echogenicity between the first and second regions of interest. After evaluating the image, the model may generate an output that is indicative or representative of whether the model predicted, detected, or otherwise determined that the image is “echogenic” (i.e., condition is present) or “non-echogenic” (i.e., condition is absent). The model may further determine the confidence level of the prediction, detection, or determination that may be in the form of a percentage. For example, if the model is certain that the image is echogenic (i.e., condition is present), the model may assign a confidence level of 100%.

Similarly, in an instance wherein the method 100 is intended to detect atrophy/fatty infiltration of a rotator cuff muscle, and first and second regions of interest of an acquired image identified in step 110 correspond to first and second anatomical structures of interest, respectively, the trained machine learning model may be configured to detect the presence or absence of atrophy/fatty infiltration based on the relative difference in the echogenicity between the first and second regions of interest. After evaluating the image, the model may generate an output that is indicative or representative of whether the model predicted, detected, or otherwise determined that the image is “echogenic” (i.e., condition is present) or “non-echogenic” (i.e., condition is absent). The model may further determine the confidence level of the prediction, detection, or determination that may be in the form of a percentage. For example, if the model is certain that the image is echogenic (i.e., condition is present), the model may assign a confidence level of 100%. Further, the model may be additionally or alternatively configured to assign an indication or grade as to the severity of the condition (e.g., mild, moderate, severe).

In an instance wherein multiple images are acquired in step 102, the evaluating step 114 and detecting step 120 may be performed separately or individually for each of the acquired images. Once steps 114 and 120 are performed for each of the images acquired in step 102, the detection, prediction, or determination of the presence or absence of the medical condition for each of the images may be combined together to detect, predict, or determine the presence or absence of the medical condition for the collection of images. In an embodiment, this may comprise evaluating the individual predictions, detections, or determinations to make an overall prediction, detection, or determination. For example, if steps 114 and 120 are performed for a plurality of images, then whatever is predicted, detected, or determined for the majority of the images may be the overall prediction, detection, or determination. So, if it is detected for a majority of the images that the medical condition is present, then the overall prediction, detection, or determination may be that the medical condition is present. Similarly, if it is detected for a majority of the images that the medical condition is absent, then the overall prediction, detection, or determination may be that the medical condition is absent.

In another embodiment, the individual predictions, detections, or determinations may be evaluated along with the confidence levels of those predictions, detections, or determinations to make an overall prediction, detection, or determination. For example, if steps 114 and 120 are performed for a plurality of images, and it is predicted, detected, or determined for every image that the medical condition is present and there is a confidence level for each prediction, detection, or determination of at least a predetermined percentage, then it may be detected, predicted, or determined that the medical condition is present. Similarly, if it is predicted, detected, or determined for every image that the medical condition is present and there is a confidence level for each prediction, detection, or determination that is below a predetermined percentage, then it may be detected, predicted, or determined that the medical condition is absent.

Yet another way an overall prediction, detection, or determination may be made for a collection of images is to combine some or all of the images acquired in step 102 to generate a single three-dimensional image using known image processing techniques. Steps 114 and 120 may then be performed on that generated three-dimensional image using, for example, a three-dimensional machine learning model to detect, predict, or determine the presence or absence of the medical condition.

Accordingly, it will be appreciated that an overall prediction, detection, or determination may be made for a collection of images in a number of ways and that the present disclosure is not intended to be limited to any particular way(s).

Whether one or more images are acquired in step 102 and evaluated in step 114, in an embodiment wherein steps 114 and 120 comprise applying a trained machine learning model, prior to applying the trained model to an image acquired in step 102, the evaluating step 114 may comprise a substep of applying one or more masks over one or more portions of the image that do not correspond to the regions of interest identified in step 110 so that the model only sees and evaluates or processes the identified regions of interest. The application of such a mask can be carried out by an electronic processor, for example, the electronic processor 12 of the system 10, using known image processing techniques.

In another embodiment, for example, one in which the method 100 includes the step 116 described above of generating one or more cropped images corresponding to the identified regions of interest prior to performing the evaluating step 114, rather than applying the trained machine learning model to the acquired image as a whole (with or without a mask applied thereto), the model may instead be applied to one or a combination of the new or cropped images generated in step 122 that correspond to the identified regions of interest. In such an embodiment, the cropped image(s) may be generated and normalized using known image processing techniques, and then the trained machine learning model may be applied thereto. For example, the cropped images may be combined into a single image (e.g., a two- or three-dimensional image) using known image processing and normalization techniques and then the trained machine learning model may be applied thereto as described elsewhere herein.

In any event, as will be appreciated by those of ordinary skill in the art, any number of trained machine learning models known in the art may be used to perform the functionality/operation described above. Suitable machine learning models or algorithms include, but are certainly not limited to: deep learning models; trained neural networks (e.g., convolutional neural networks (CNN)); three-dimensional machine learning models, and object classification models/algorithms. For purposes of illustration only, one particular model or algorithm that may be used is the VGG-19 convolutional neural network. It will be appreciated that to use such a model, the images to which the model is applied may have to be resized (e.g., to 224×224) and/or cropped to meet the sizing requirement of the model, and in an instance wherein grayscale images are used (e.g., ultrasound images), the image may have to be copied to generate the 3-channel image required by the model. While one specific model is identified and described above, it will be appreciated that a number of other suitable models or algorithms may certainly be used instead.

Regardless of the particular trained machine learning model or algorithm that is used, it may be trained using techniques well known in the art. While the particular way the model is trained may be model-dependent, in general terms, a first set of images (i.e., training images) are fed to the model. These images may be tagged or otherwise marked as being representative of either the presence of the medical condition or the absence of the medical condition. The model then learns to recognize both the presence and absence of the medical condition from the training images and works to recognize them using a second set of images (i.e., test images) that may or may not include one or more of the first set of training images. Based on the performance of the model with the test images, parameters of the model (e.g., biases, weights, etc.) may be adjusted or tuned to improve performance.

It is to be understood that the foregoing is a description of one or more embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Further, the terms “electrically connected” or “electrically coupled” and variations thereof are intended to encompass both wireless electrical connections and electrical connections made via one or more wires, cables, or conductors (wired connections). Other terms are to be construed using their broadest reasonable meaning unless they are used in a context that requires a different interpretation.

1. A method of detecting the presence or absence of a particular medical condition for a patient, comprising:
acquiring at least one image of an area of interest of the patient's body;
identifying a first region of interest within the at least one acquired image corresponding to a first anatomical structure of interest and a second region of interest within the at least one acquired image corresponding to a second anatomical structure of interest;
evaluating the first and second regions of interest;
detecting the presence or absence of the medical condition based on the evaluation of the first and second regions of interest; and
generating an electrical signal indicative of the detected presence or absence of the medical condition.

2. The method of claim 1, further comprising outputting the electrical signal to a display to cause an indication representative of the detection of the presence or absence of the medical condition to be provided.

3. The method of claim 1, wherein the evaluating step comprises calculating a score for each of the at least one image based on a given parameter of the first region of interest and the second region of interest, and the detecting step comprises detecting the presence or absence of the medical condition based on the score.

4. The method of claim 3, wherein the score comprises a ratio of the given parameter of the first region of interest to the given parameter of the second region of interest.

5. The method of claim 3, wherein the first anatomical structure of interest comprises a bone of the patient's body, and the second anatomical structure of interest comprises a muscle of the patient's body.

6. The method of claim 1, wherein the identifying step comprises applying a trained machine learning model to the at least one acquired image to identify the first and second regions of interest, wherein the trained machine learning model is trained to identify the first region of interest by recognizing the first anatomical structure of interest in the acquired image and to identify the second region of interest by recognizing the second anatomical structure of interest in the acquired image.

7. The method of claim 1, wherein following the identifying step and before the evaluating step, the method comprises generating a first cropped image corresponding to the first region of interest and a second cropped image corresponding to the second region of interest, and further wherein the evaluating step comprises evaluating the first and second cropped images.

8. The method of claim 1, wherein the acquiring step comprises acquiring a plurality of images of the area of interest, and further wherein the identifying and evaluating steps are performed for two or more of the plurality of acquired images, and the detecting step comprises detecting the presence or absence of the medical condition based on the evaluation of the first and second regions of interest of the two or more of the plurality of acquired images.

9. The method of claim 8, wherein for each of the two or more of the plurality of acquired images, the evaluating step comprises calculating a score based on a given parameter of the first region of interest and the second region of interest of that image, and further wherein the method comprises determining a combined score for the two or more of the plurality of images based on the scores determined for each of the two or more of the plurality of images, and further wherein the detecting step comprises detecting the presence or absence of the medical condition based on the combined score.

10. The method of claim 1, wherein the evaluating step comprises applying a trained machine learning model to at least portions of the at least one acquired image corresponding to the identified first and second regions of interest, wherein the trained machine learning model is trained to detect the presence or absence of the medical condition based on the first and second regions of interest.

11. A system for detecting the presence or absence of a particular medical condition for a patient, comprising:
one or more electronic processors; and
one or more electronic memories each electrically connected to at least one of the one or more electronic processors and having instructions stored therein;
wherein the one or more electronic processors are configured to access the one or more electronic memories and to execute the instructions stored therein such that the one or more electronic processors are configured to:
acquire at least one image of an area of interest of the patient's body;
identify a first region of interest within the at least one acquired image corresponding to a first anatomical structure of interest and a second region of interest within the at least one acquired image corresponding to a second anatomical structure of interest;
evaluate the first and second regions of interest;
detect the presence or absence of the medical condition based on the evaluation of the first and second regions of interest; and
generate an electrical signal indicative of the detected presence or absence of the medical condition.

12. The system of claim 11, wherein the system further comprises a display and the one or more electronic processors are further configured to output the electrical signal to a display to cause an indication of the detection of the presence or absence of the medical condition to be provided.

13. The system of claim 11, wherein the one or more electronic processors is configured to evaluate the first and second regions of interest by calculating a score for each of the at least one image based on a given parameter of the first region of interest and the second region of interest, and to detect the presence or absence of the medical condition based on the score.

14. The system of claim 13, wherein the score comprises a ratio of the given parameter of the first region of interest to the given parameter of the second region of interest.

15. The system of claim 11, wherein the one or more electronic processors are configured to identify the first and second regions of interest by applying a trained machine learning model to the at least one acquired image to identify the first and second regions of interest, wherein the trained machine learning model is trained to identify the first region of interest by recognizing the first anatomical structure of interest in the acquired image and to identify the second region of interest by recognizing the second anatomical structure of interest in the acquired image.

16. The system of claim 11, wherein the one or more electronic processors are further configured to generate a first cropped image containing the first region of interest and a second cropped image containing the second region of interest, and further wherein the one or more electronic processors are configured to evaluate the first and second regions of interest by evaluating the first and second cropped images.

17. The system of claim 11, wherein the one or more or electronic processors are configured to acquire a plurality of images of the area of interest, and further wherein the one or more electronic processors are configured to identify the first and second regions of interest and to evaluate the first and second regions of interest for two or more of the plurality of acquired images, and the one or more electronic processors are configured to detect the presence or absence of the medical condition based on the evaluation of the first and second regions of interest of the two or more of the plurality of acquired images.

18. The system of claim 20, wherein for each of two or more of the plurality of acquired images, the one or more electronic processors are configured to evaluate the first and second regions of interest by calculating a score based on a given parameter of the first region of interest and the given parameter of the second region of interest, and further wherein the one or more electronic processors is configured to determine a combined score for the two or more of the plurality of images based on the scores determined for each of the two or more of the plurality of images, and to detect the presence or absence of the medical condition based on the combined score.

19. The system of claim 11, wherein the one or more electronic processors are configured to evaluate the first and second regions of interest by applying a trained machine learning model to at least portions of the at least one acquired image corresponding to the identified first and second regions of interest, wherein the trained machine learning model is trained to detect the presence or absence of the medical condition based on the first and second regions of interest.

20. A non-transitory, computer-readable storage medium storing program instructions thereon that, when executed on one or more electronic processors, causes the one or more electronic processors to carry out the method of:
acquiring at least one image of an area of interest of the patient's body;
identifying a first region of interest within the at least one acquired image corresponding to a first anatomical structure of interest and a second region of interest within the at least one acquired image corresponding to a second anatomical structure of interest;
evaluating the first and second regions of interest;
detecting the presence or absence of the medical condition based on the evaluation of the first and second regions of interest; and
generating an electrical signal indicative of the detected presence or absence of the medical condition.
